(19) United States
(12) Patent Application Publication
(10) Pub. No.: US 2005/0148567 A1
(43) Pub. Date: Jul. 7, 2005

(54) Treatment of tattoos by photodynamic therapy

(76) Inventors: Charles R. Kjellbotn, Surrey (CA); Philippe Maria C. Margaron, Burnaby (CA); Patricia J. McNicol, Montreal (CA); John R. North, Vancouver (CA)

Correspondence Address: MORRISON & FOERSTER LLP, 3811 VALLEY CENTRE DRIVE, SUITE 500, SAN DIEGO, CA, 92130-2332, US

(21) Appl. No.: 10/922,621

(22) Filed: Aug. 20, 2004

(30) Foreign Application Priority Data
Aug. 20, 2003 (CA) 2437638

(51) Int. Cl. A61K 31/555; A61K 31/5415; A61K 31/198; A61K 31/409; A61N 1/30

(52) U.S. Cl. 514/185; 514/410; 514/224.8; 604/20; 514/561

(57) ABSTRACT

The present invention relates to a photodynamic method of treating tattoos. The method comprises: (i) intradermally and/or locally delivering photosensitizer into tattooed target tissue; and (ii) irradiating the target tissue with activation energy at a wavelength appropriate to activate the photosensitizer. The present method causes the tattoo inks to fade or disappear completely. In preferred embodiments the tattoo will fade by at least 25%, more preferably at least 50%, even more preferably at least 75%.

### RELATED PATENT APPLICATIONS

This application claims priority to Canadian Patent Application no. 2437638 filed 20 Aug. 2003 which is incorporated in its entirety herein by reference.

### FIELD OF THE INVENTION

The present invention relates to photodynamic therapy (PDT). In particular, the present invention relates to photodynamic methods, compositions, and devices for the treatment of tattoos.

### BACKGROUND OF THE INVENTION

Tattooing is an invasive procedure where pigments, typically permanent ones, are introduced into the skin. Dating back to at least the ancient Egyptians, tattooing has been documented in a variety of cultures and for a variety of motivations. For example, the tattoos of New Zealand's pre-colonial Maori population were both decorative and an expression of an individuals legal identity. Indeed, in the early days of the colonial era, Maoris would often sign European documents by painstakingly drawing their entire facial design. In modern-day western culture, the cultural status of tattooing has steadily evolved from that of an anti-social, rebellious activity confined largely to sailors and jailers, in the 1960s to a trendy fashion statement in the present day. First adopted and flaunted by influential rock stars like the Rolling Stones in the early 1970s, tattooing has become accepted by ever broader segments of society until today when tattoos are routinely seen on rock stars, professional athletes, fashion models, movie stars and college students.

Professional tattooing usually involves pigment being injected into the skin via a vertically vibrating needle. The subject typically receives between 50 to 3000 needle punctures per minute which drives the pigment into the dermis. In recent years tattoos have grown markedly in popularity particularly among teenagers and those in their early twenties. However, this increase in popularity has led to a concurrent increase in the demand for removal of these youthful indiscretions. Unwanted or inappropriate tattoos can have a large psychological impact and can cause embarrassment and low self-esteem. Some choose to cover the tattoo with make-up, clothes or adhesive bandages but many would prefer a more permanent removal.

Currently, the treatments for removal of tattoos are rather limited. Options include excision, dermabrasion and salabrasion, all of which can be painful, can cause scarring, and are not always efficacious. A more commonly used treatment is laser removal. This entails delivering light energy to the tattoo in order to break the pigments into fragments which are then removed by the subjects' immune system. The advantages of laser removal over the surgical or abrasive techniques are obvious. However, laser removal can be expensive, painful, is not always efficacious, and requires different lasers to treat all pigment colours. In addition, the laser light, particularly with short pulse Q-switched lasers, can cause reactions in certain of the chemicals used in the inks leading to permanent darkening. Furthermore, dark colours such as blue or black respond better to the treatment than light colours such as green or yellow.

There exists a need for an efficacious therapy for removing or fading tattoos. Preferably, any therapy would address one or more of the issues identified above.

Photodynamic therapy (PDT) involves delivery of a photosensitive agent to a target tissue and activation of that agent with an appropriate energy source. Clinical trials have been conducted testing PDT as a potential therapy for various indications including squamous cell carcinoma, basal cell carcinoma, actinic keratosis, age-related macular degeneration, and Barrett's esophagus. It has also been proposed that PDT may be an effective treatment in many other indications. See, for example, U.S. Pat. No. 5,095,030 (Levy et al.) which lists typical indications as including destruction of solid tumors, dissolution of plaques in blood vessels, treatment of topical indications such as acne, athletes foot, warts, papilloma, psoriasis, and the treatment of biological products such as blood for infectious agents. U.S. Pat. No. 6,171,332 (Whitehurst) relates to a cosmetic method of treatment of dermatological conditions by irradiating the affected area with an incoherent high-intensity non-laser light beam having an intensity of greater than 0.075 watts per cm², the light beam having a bandwidth in the range 0 to 30 nm. This reference mentions portwine stains, tattoos and psoriasis as potential dermatological conditions to be treated.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents. Unless otherwise specified, all documents referred to herein as incorporated by reference in their entirety.

### SUMMARY OF THE INVENTION

The present invention relates to a photodynamic method of treating tattoos. The method comprises:

(i) intradermally and/or locally delivering photosensitizer directly into tattooed target tissue; and

(ii) irradiating the target tissue with activation energy at a wavelength appropriate to activate the photosensitizer.

While not wishing to be bound by theory, it is believed that the photodynamic therapy causes the fragmentation of tattoo ink particles, possibly by disrupting the ink-loaded dermal cells, which results in the release of the ink particles. A local inflammatory reaction is then believed to clear the ink particles. It has been found that the present method can effectively fade or remove various colours of tattoos including, but not limited to, green, blue, and black.

As used herein “intradermally” or “intradermal” means administering photosensitizer through the stratum corneum directly to the target tissue. Any suitable means of causing the photosensitizer to penetrate the stratum corneum may be used. For example, intradermal administration can be via an injection directly into the dermal tissue. Or by topical application of a composition that causes the photosensitizer to penetrate the stratum corneum. Or by use of a device that facilitates the penetration of photosensitizer through the stratum corneum.

In preferred embodiments, the photosensitizer is delivered primarily to the site of the tattoo. Typically, tattoo inks reside in the dermal tissue.

### BRIEF DESCRIPTION OF THE DRAWINGS

FIG. 1 shows examples of the tattoo response scoring scale.

### DETAILED DESCRIPTION OF THE INVENTION

The present invention relates to a photodynamic method of treating tattoos. The method comprises:

(i) intradermally delivering photosensitizer into tattooed target tissue; and

(ii) irradiating the target tissue with energy at a wavelength appropriate to activate the photosensitizer.

Another aspect of the present invention relates to a photodynamic method of treating tattoos. The method comprises:

(i) locally delivering photosensitizer into tattooed target tissue; and

(ii) irradiating the target tissue with energy at a wavelength appropriate to activate the photosensitizer.

The present method causes the tattoo inks to fade or disappear completely. In preferred embodiments the tattoo will fade by at least 25%, more preferably at least 50%, even more preferably at least 75%, as assessed according to the test method described below.

In order to assess the amount of fading of a tattoo, photographs are taken prior to treatment using a camera set-up designed to ensure a standard view of the tattoos. For example, an Olympus SZX9 Dissecting scope with DP12 camera and 0.3× lens with the magnification set at 2.1 and a ring light NCL150 attached to a high intensity light source may be used. After the course of treatment the skin is allowed to heal and the tattoo is photographed again. The photographs are then assessed by at least two independent, blinded Assessors. The Assessors score the tattoo response to PDT in accordance with the following scale:

GRADE 1—Tattoo not visibly altered from original tattoo

GRADE 2—Tattoo slightly faded. Edges blurry/indeterminate. Size of tattoo not significantly altered from original (<25%)

GRADE 3—Tattoo visibly faded. Edges blurry/indeterminate. Size of tattoo visibly altered <50%

GRADE 4—Tattoo predominately faded. Edges blurry/indeterminate. Size of tattoo altered >50%

GRADE 5—Tattoo predominately faded. Tattoo difficult to distinguish but pigment still visible. Gaps in tattooed area are apparent (i.e., patchy pigment)

GRADE 6—Tattoo completely faded. Tattooed area difficult to distinguish from normal tissue

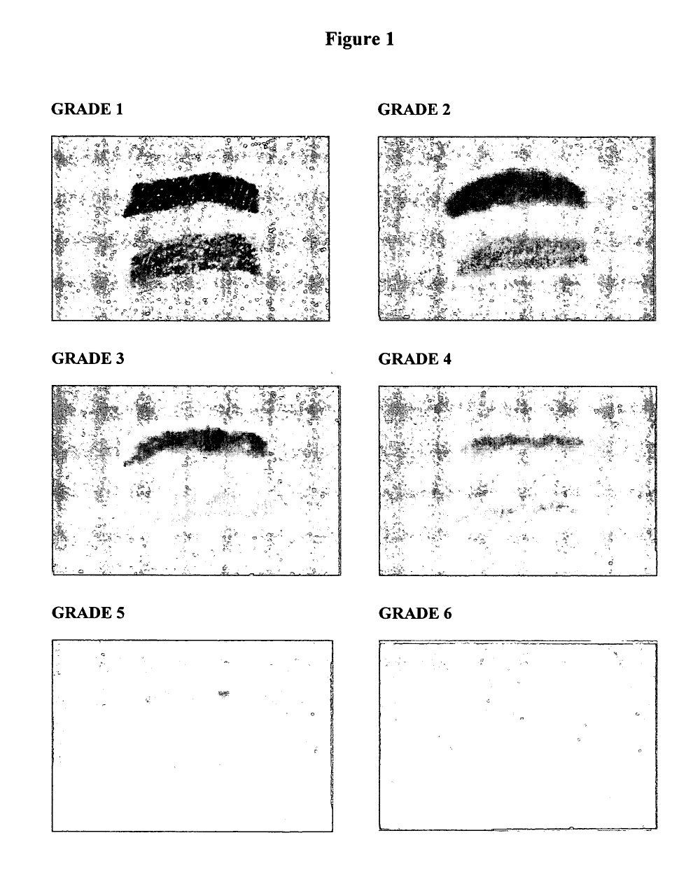

FIG. 1 shows an example of the visual scale used to grade the tattoo fading. After grading the median tattoo response score is used to determine the level of fading.

The present method can be a cosmetic method of treatment.

In the present method, the photosensitizer is delivered intradermally. Any suitable means of intradermal delivery may be used. Preferred means include, but are not limited to, injection by needle, needleless pressure-injection, topical delivery, iontophoresis, tattoo gun, and combinations thereof. Examples of suitable needle injection devices include, but are not limited to, needles and syringe combinations of varying sizes. While delivery via a needle works well, it is believed that a needleless delivery system would offer certain advantages. For example, such systems are said to be less painful than using a needle and there is no needle that might become blunt. Examples of suitable needle injection devices include, but are not limited to, Dermo-Jet™ (Robbins Instruments, Chatham, N.J. 07928, USA), PowderJect™ (PowderJect Pharmaceuticals Plc, Oxford, 0X4 4GA, England), Penjet™ (PenJet Corporation, Beverly Hills, Calif. 90212, USA), Injex™ (Equidyne Systems Inc, San Diego, Calif. 92121, USA), and Biojector™ (Bioject Inc, Bedminster, N.J. 07921, USA). Certain of these devices may require some modification before they are adapted to provide appropriate intradermal injections. Other devices such as the PassPort system (available from Altea Therapeutics, Tucker, Ga., USA and described in WO03/77971 and WO03/101507) or the Macroflux system (available from Alza, Mountain View, Calif., USA) may be used to deliver photosensitizer into the skin. Alternatively, ionophoresis methods such as the E-trans system (available from Alza, Mountain View, Calif., USA) may be used.

Preferably, the photosensitizer herein is delivered to the target such that an adequate concentration of photosensitizer is found in the tissues containing the tattoo inks. Preferably, there are not significant amounts of photosensitizer on the skin surface. It is thought that if there is a large amount of photosensitizer activated at the skin surface during the irradiation step it may cause unwanted destruction of skin tissue. In addition, it may prevent the activation energy from activating the photosensitizer at the target tissue.

It is preferred that the peak concentration of photosensitizer is at a depth of at least about 0.5 mm, more preferably at least about 1 mm, even more preferably at least about 1.5 mm, from the surface of the skin.

The amount of photosensitizer used will be determined by a variety of factors such as the type of photosensitizer, the activation energy, the type/colour of tattoo, the depth of the tattoo, the size of the tattoo, the age of the tattoo, the skin type/colour, the location of the tattoo etc. While it will be understood that the dosage varies greatly depending on these factors, typical doses include, for example, from about 0.1 μg of photosensitizer per cm² of treatment area to about 1 g/cm², preferably about 1 μg/cm² to about 1 mg/cm², more preferably from about 10 μg/cm² to about 500 μg/cm².

As used herein, “photosensitizer” or “photosensitizing agent” means a compound, or precursor of a compound, which, when contacted by radiation, induces fading or removal of tattoos. For clarity, it is intended that this definition include pro-drugs such as ALA or ALA-esters as well as preformed photosensitizing agents such as verteporfin. Preferably, the compound is nontoxic to humans or is capable of being formulated in a nontoxic composition. Preferably, the compound in its photodegraded form is also nontoxic. A non-limiting listing of photosensitive chemicals may be found in Kreimer-Birnbaum, Sem. Hematol. 26:157-73, 1989 (incorporated herein by reference) and in Redmond and Gamlin, Photochem. Photobiol. 70 (4):391-475 (1999).

There are a variety of preferred synthetic and naturally occurring photosensitizers, including, but not limited to, pro-drugs such as the pro-porphyrin 5-aminolevulinic acid (ALA) and derivatives thereof, porphyrins and porphyrin derivatives e.g., chlorins, bacteriochlorins, isobacteriochlorins, phthalocyanine and naphthalocyanines and other tetra- and poly-macrocyclic compounds, and related compounds (e.g., pyropheophorbides, sapphyrins and texaphyrins) and metal complexes (such as, but not limited by, tin, aluminum, zinc, lutetium). Tetrahydrochlorins, purpurins, porphycenes, and phenothiaziniums are also within the scope of the invention. Other suitable photosensitizers include bacteriochlorophyll derivatives such as those described in WO-A-97/19081, WO-A-99/45382 and WO-A-01/40232. A preferred bacteriochlorophyll is palladium-bacteriopheophorbide WST09 (Tookad™). Preferably the photosensitizers are selected from pro-porphyrins, porphyrins, and mixtures thereof. Some examples of pro-drugs include aminolevulinic acid such as Levulan™ and aminolevulinic acid esters such as described in WO-A-02/10120 and available as Metvix™, Hexvix™ and Benzvix™. Some examples of di-hydro or tetra-hydro porphyrins are described in EP-A-337,601 or WO-A-01/66550 and available as Foscan™ (temoporfin). Combinations of two or more photosensitizers may be used in the practice of the invention. Some examples of suitable compounds include, but are not limited to, those described in U.S. Pat. Nos. 6,462,192; 6,444,194; 6,376,483; WO-A-03/028628; WO-A-03/028629; WO-A-02/096417; and WO-A-02/096366, all of which are herein incorporated by reference.

Preferably, the photosensitizer for use herein is selected from one or more of aporphyrin precursor, a porphyrin, a porphyrin derivative, a phenothiazinium, a bacteriochlorophyll, aminolevulinic acid, aminolevulinic acid derivative, and combinations thereof. More preferably, the photosensitizer for use herein is selected from a porphyrin derivative, phenothiazinium, and combinations thereof.

In one embodiment it is preferred that the photosensitizer is selected from those which photobleach upon exposure to activation energy.

In preferred embodiments of the invention, the photosensitizer is selected from a group of photosensitizers known as green porphyrins. The term “green porphyrins” refers to porphyrin derivatives obtained by reacting a porphyrin nucleus with an alkyne in a Diels-Alder type reaction to obtain a mono-hydrobenzoporphyrin. Such resultant macropyrrolic compounds are called benzoporphyrin derivatives (BPDs), which is a synthetic chlorin-like porphyrin with various structural analogues, as shown in U.S. Pat. No. 5,171,749 (incorporated herein by reference). Typically, green porphyrins are selected from a group of tetrapyrrolic porphyrin derivatives obtained by Diels-Alder reactions of acetylene derivatives with protoporphyrin under conditions that promote reaction at only one of the two available conjugated, nonaromatic diene structures present in the protoporphyrin-IX ring systems (rings A and B). Metallated forms of a Gp, in which a metal cation replaces one or two hydrogens in the center of the ring system, may also be used in the practice of the invention. The preparation of the green porphyrin compounds useful in this invention is described in detail in U.S. Pat. No. 5,095,030 (hereby incorporated by reference).

Preferably, the BPD is a benzoporphyrin derivative diester di-acid (BPD-DA), mono-acid ring A (BPD-MA), mono-acid ring B (BPD-MB), or mixtures thereof. These compounds absorb light at about 692 nm wavelength and have improved tissue penetration properties. The compounds of formulas BPD-MA and BPD-MB may be homogeneous, in which only the C ring carbalkoxyethyl or only the D ring carbalkoxyethyl would be hydrolyzed, or may be mixtures of the C and D ring substituent hydrolyzates. A number of other BPD B-ring derivatives may also be used in the present methods. These derivatives have the following general formula:

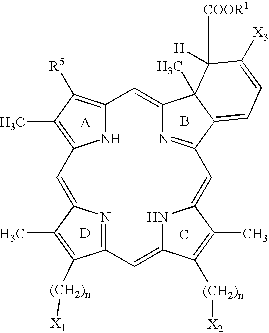

wherein; R⁵ is vinyl, R¹ and R⁶ are methyl, and n is 2. X₁, X₂, and X₃ are listed in the tables below:

TABLE 1
Hydrophilic BPD B-ring analogs

| Drug | X1 | X2 | X3 |
|---|---|---|---|
| QLT0061 | COOH | COOH | COOH |
| QLT0077 | CONH(CH₂)₂N⁺(CH₃)₃I⁻ | CONH(CH₂)₂N⁺(CH₃)₃I⁻ | COOCH³ |
| QLT0079 | CONH(CH₂)₂N + (CH₃)₂((CH2)₃CH₃ | CONH(CH₂)₂N⁺(CH₃)₂((CH2)₃CH³) | COOCH³ |
| QLT0086 | CONHCH(COOH)CH2COOH | CONHCH(COOH)CH₂COOH | COOCH³ |
| QLT0092 | CONH(CH2)₂NH(CH₃)₂CF₃COO⁻ | CONH(CH2)₂NH(CH₃)₂CF₃COO⁻ | COOCH₃ |
| QLT0094 | CONHCH₂COOH | CONHCH²COOH | CONHCH²COOH |

TABLE 2
Lipophilic BPD B-ring analogs

| Drug | X1 | X2 | X3 |
|---|---|---|---|
| QLT0060 | CO(O(CH₂)₂)OH | CO(O(CH2)2)0H | COOCH₃ |
| QLT0069 | COOCH₃ | COOCH₃ | COOH |
| QLT0078 | CO(O(CH₂)₂)₂OH | CO(O(CH₂)₂)₂OH | COOCH₃ |
| QLT0080 | CO(O(CH₂)₂)₃OH | CO(O(CH₂)₂)₃OH | COOCH₃ |
| QLT0081 | CO(O(CH₂)₂)₂)OCH3 | CO(O(CH₂)₂)₂OCH3 | CO(O(CH₂)₂)₂OCH₃ |
| QLT0082 | CO(O(CH₂)₂)₂OH | CO(O(CH₂)₂)₂OH | CO(O(CH₂)₂)₂OH |
| QLT0083 | CO(O(CH₂)₂)₃OH | CO(O(CH₂)₂)₃OH | CO(O(CH₂)₂)₃OH |
| QLT0087 | CO(O(CH₂)₂)₄OH | CO(O(CH₂)₂)₄OH | COOCH₃ |
| QLT0088 | COOCH₃ | COOCH₃ | CONH(C₆H₄)(C₅H₁₀N) |
| QLT0090 | CO(O(CH₂)₂)₅OH | CO(O(CH2)₂)₅OH | COOCH₃ |
| QLT0093 | CO(O(CH₂)₂)₅OH | CO(O(CH2)₂)₅OH | CO(O(CH₂)₂)₅OH |

Preferred photosensitizers are the benzoporphyrin derivative mono-acid (BPD-MA), QLT0074 (as set forth in U.S. Pat. No. 5,929,105 referred to therein as A-EA6) and B3 (as set forth in U.S. Pat. No.5,990,149). Most preferred for use herein is QLT0074 which has the structure:

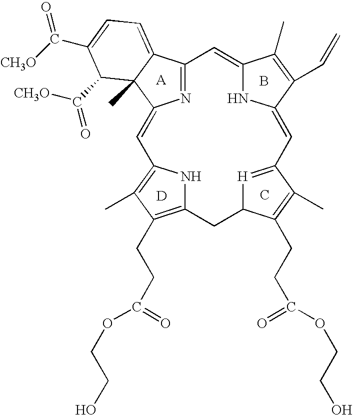

Additionally, the photosensitizers used in the invention may be conjugated to various ligands to facilitate targeting. These ligands include receptor-specific peptides and/or ligands as well as immunoglobulins and fragments thereof. Preferred ligands include antibodies in general and monoclonal antibodies, as well as immunologically reactive fragments of both.

Dimeric forms of the green porphyrin and dimeric or multimeric forms of green porphyrin/porphyrin combinations can be used. The dimers and oligomeric compounds of the invention can be prepared using reactions analogous to those for dimerization and oligomerization of porphyrins per se. The green porphyrins or green porphyrin/porphyrin linkages can be made directly, or porphyrins may be coupled, followed by a Diels-Alder reaction of either or both terminal porphyrins to convert them to the corresponding green porphyrins. Of course combinations of two or more photosensitizers may be used in the practice of the invention.

In addition to the above mentioned preferred photosensitizing agents, other examples of photosensitizers useful in the invention include, but are not limited to, green porphyrins disclosed in U.S. Pat. Nos. 5,283,255, 4,920,143, 4,883,790, 5,095,030, and 5,171,749; and green porphyrin derivatives, discussed in U.S. Pat. Nos. 5,880,145 and 5,990,149 (all of which are incorporated by reference). Several structures of typical green porphyrins are shown in the above cited patents, which also provide details for the production of the compounds.

Once the photosensitizer has been delivered to the target tissue it can be activated by any suitable energy source in any suitable manner. It is preferred that the activation energy is delivered directly to the skin above the tattoo. Therefore, it is preferred that the delivery device be adapted or adaptable to deliver activation energy directly to the skin in a relatively uniform manner.

The time between administration of photosensitizer and administration of activation energy will vary depending on a number of factors. Activation energy delivery can take place at any suitable time following administration of photosensitizer as long as there is still photosensitizer present at the skin. Activation energy treatment within a period of about one minute to about 1 week after administration of the photosensitizer is preferred, with a range of 5 minutes to 6 hours being more preferred. However, some photosensitizers, such as ALA and ALA-ester may require a longer period as they must be converted into the active compound within the target tissue before treatment can proceed.

The activation energy should be capable of penetrating the tissue to a depth sufficient to activate the PS at the target tissue. In general, the longer the wavelength of the activation energy, the greater the penetration. Preferably, the activation energy penetrates at least 1 mm, more preferably at least 2 mm, even more preferably at least 3 mm into the skin.

Preferably the activation energy has a wavelength of from about 380 nm to about 900 nm, more preferably from about 400 nm to about 800 nm, even more preferably from about 450 nm to about 750 nm. Preferably, the activation energy comprises a wavelength close to at least one of the absorption peaks of the photosensitizer(s) used. This wavelength differs for different photosensitizers. For example, BPD-MA has an absorption peak at 692 nm and so, when BPD-MA is the photosensitizer used, the wavelength of the activation energy preferably is at or close to 692 nm. The photosensitizers ALA (available under the tradename Levulan) and ALA-methyl ester (available under the tradename Metvix) have several absorption peaks including those at around 400-440 nm and another at around 630 nm so when these photosensitizer are used the activation energy is preferably at or close to 400-440 (such as provided by the BLU-U™ light source) and/or 630 nm (such as provided by the Aktilite™ light source).

Preferably the activation energy has a full-width half-maximum (FWHM) of less than 100 nm, more preferably less than 75 nm, even more preferably less than 50 nm.

Any appropriate activation energy source, depending on the absorption spectrum of the photosensitizer, may be used for photosensitizer activation. Preferred sources include, but are not limited to, lasers, light emitting diodes (LED), incandescent lamps, arc lamps, standard fluorescent lamps, U.V. lamps, and combinations thereof. More preferred are lasers, light emitting diodes, or combinations thereof. Alternatively, any convenient source of activation energy having a component of wavelengths that are absorbed by the photosensitizer may be used, for example, an operating room lamp, or any bright light source, including sunlight. The activation energy dose administered during the PDT treatment contemplated herein can vary as necessary. Preferably, for photosensitizers of high potency, such as green porphyrins, the dosage of the light is typically from about 1 to about 200 J/cm². It is generally preferred that the total dose of the irradiation should generally not exceed 200 J/cm², or more preferably not exceed 100 J/cm². Preferred doses range between about 0.01 J/cm² to about 200 J/cm², more preferably 0.1 J/cm² to about 100 J/cm². For example, the dose can be about 1, about 5, about 10, about 15, about 20, about 25, or about 30 J/cm². More preferred doses range from about 5 J/cm² to about 25 J/cm².

Commercially available activation energy sources include Aktilite™, CureLight™ (both available from Photocure ASA, Oslo, Norway), BLU-U™ (available from DUSA, Wilmington, Mass., USA), PDT Laser (available from Diomed, Andover, Mass., USA), Ceralas™ (available from Biolitec AG, Jena, Germany), Q-Beam & Quanta-med and Quantum Devices (e.g., Q-100) LED Panel (Quantum Devices Inc, Barneveld Wis., USA).

Wavelengths in the ultraviolet range should, however, generally be avoided because of their mutagenic potential. It is preferred that the activation energy used for the methods herein is not in the ultraviolet range.

The activation energy dose administered during the PDT treatment contemplated herein can vary as necessary. Preferably, for photosensitizers of high potency, such as green porphyrins, the dosage of the light is typically from about I to about 200 J/cm². It is generally preferred that the total dose of the irradiation should generally not exceed 200 J/cm², or more preferably not exceed 100 J/cm². Preferred doses range between about 0.01 J/cm² to about 200 J/cm², more preferably 0.1 J/cm² to about 100 J/cm². For example, the dose can be about 1, about 5, about 10, about 15, about 20, about 25, or about 30 J/cm². More preferred doses range from about 5 J/cm² to about 25 J/cm².

The intensity of the energy source preferably does not exceed about 2000 mW/cm². Preferably, irradiances of between about 10 and 400 mW/cm², and more preferably between 25 and 75 mW/cm², are used

Preferably, the irradiation lasts from about 10 seconds to about 4 hours, more preferably between about 30 seconds to about 60 minutes, even more preferably between about 1 minutes and 30 minutes. The irradiation time is dependent on many factors and so can vary considerably. For example, irradiation times of about 1, about 2, about 3, about 4, about 5, about 6, about 7, about 8, about 9, about 10, about 15, about 20, about 30, about 45, and about 60 minutes may be used.

While not wishing to be bound by theory, it is believed that different photosensitizers and different activation energies will require different parameters in order to cause fading of the tattoo. Such parameters can be determined by simple dose-ranging studies. For example, a suitable method could involve:

(a) assessing the tattoo,

(b) intradermally delivering various concentrations of photosensitizer to the tattooed tissue,

(c) waiting for varying lengths of time,

(d) treating with various activation energy doses, and

(e) assessing the level of fading after a suitable interval.

It is preferred that the present method not involve a PDT dose that results in extensive cell death and tissue disruption in the treatment area.

It is preferred that the area to be treated have minimal hair coverage when the activation energy is applied. Therefore, if there is significant hair coverage in the area to be treated, it is preferred that the hair is shaved prior to activation energy application.

The irradiation or light exposure used in the invention may be directed to a small or large area of the body or scalp depending on the size of tattoo to be treated.

Preferably the tattoo is treated as many times as necessary to achieve the desired result. The desired result may be achieved by a single treatment but usually two or more treatments are necessary. It is preferred that the total number of treatments be from 1 to 12, more preferably from 1 to 6. Preferably, if the treatment is repeated, at least one week, more preferably at least two weeks, even more preferably at least three weeks, is left between treatments. It is believed that the PDT treatment causes an eschar or scab to form over the target area. It is preferred that the area is only retreated once the tissue has healed and the scab/eschar has been removed.

A preferred regimen according to the present invention comprises:

(a) intradermally administering photosensitizer to tattooed skin. The preferred photosensitizer is QLT0074 and the preferred dose is from about 10 μg/cm² to about 500 μg/cm².

(b) administering activation energy which preferably has a wavelength of from 400 nm to 800 nm. Preferably the activation energy is delivered from an LED, laser or combinations thereof.

(c) repeating the treatment two or more times. Preferably at least three weeks is left between each treatment.

### EXAMPLES

It will be understood that the following embodiments of the present invention are intended to be illustrative of some of the possible applications or principles. Various modifications may be made by the skilled person without departing from the true spirit and scope of the invention.

### Example 1

Five guinea pigs were lightly anesthetized and shaved. Depilation of remaining hair was carried out using Nair®. Tattoos were applied using the Aims IIIA Tattoo Identification System with a 3-prong needle (Aims Inc, Hornell N.Y.) set at a penetration depth of 1 mm. Tattoo lines were applied side by side to create a rectangular filled area approximately 3×10 mm. Black ink (pigment #242, Aims Inc, Hornell N.Y.) and green/blue ink (pigment #270, Aims Inc, Hornell N.Y.) were used.

Three pairs of rectangles approximately 3×10 mm each were tattooed on each flank of each animal for a total of 6 tattoo sites on each flank. One tattoo of each pair was created using black ink, the other using green/blue ink. The tattoo pairs were side by side, at least 1 cm apart, on the animal's flank as shown in FIG. 1. The pattern was repeated on the opposite flank.

QLT0074 for injection (A-EA6 in U.S. Pat. No. 5,929,105) was reconstituted with Water for Injection to give a stock concentration of 2.0 mg/ml and then diluted with 5% Dextrose in water to a concentration of 0.1 mg/ml, 0.2 mg/ml or 1.0 mg/ml. 6±2 injections to give a total volume of 100 μL of QLT0074 or QLT0074 vehicle were injected intradermally across each pair of tattoos using a syringe and a 26 gauge ⅜ long needle. Group 1 received 0.1 mg/ml, Group 2 received 0.2 mg/ml and Group 3 received 1.0 mg/ml. In addition, the control group received an intradermal injection of the QLT0074 vehicle without photosensitizer diluted 1/50 with 5% Dextrose in water. Injections were spaced to provide approximately uniform coverage of drug across tattooed area. Excess drug was removed from the treatment site immediately after drug delivery using gauze.

Fifteen minutes after injection of the drug, the skin was exposed to 10 J/cm² of LED-generated red light (688 nm-Q-100 LED Panel (Quantum Devices Inc, Barneveld Wis., USA)) at 75 mW/cm².

PDT was repeated twice on each guinea pig at 23 and 26 days after the first and second treatments, respectively, once the skin at the treatment sites was deemed sufficiently healed.

Skin response scoring was monitored on days 1, 3, 7 and 14 post PDT and then at least weekly until the end of the study. After repeat PDT treatments the same schedule was also followed.

Photographs were taken (Olympus SZX9 Dissecting scope with DP12 camera and 0.3× lens) on the day prior to PDT, days 1, 3, 7 and 14 post PDT and at least weekly until the end of the study. After repeat PDT treatments the same schedule was also followed. The magnification was set at 2.1 and the ring light NCL150 to high with an intensity of four. This ensured a standard view of the tattoos that completely fills the image frame and provides consistent lighting.

Guinea pigs were scored by two independent assessors who were masked to the treatments. They evaluated skin response to PDT on day 1, 3, 7 and 14 post PDT then at least weekly. The scores were assessed in accordance with Table 3.

TABLE 3

| | Erythema and Eschar Formation |
|---|---|
| 0 | No observable reaction |
| 1 | Hardly detectable |
| 2 | Slight - visible pale pink, no vessels broken, no red spots |
| 3 | Blanching - few broken vessels, no eschar formation |
| 4 | Erythema - more broken vessels, leading to yellow eschar formation |
| 5 | Severe - many broken vessels, eschar formation - but less than 50% |
| | of site |
| 6 | Very severe - rosette, eschar formation on more than 50% of site |
| | Edema |
| 1 | Slight within exposure site |
| 2 | Mild within exposure site |
| 3 | Moderate |
| 4 | Severe - extending beyond exposure side |

The sum of scores from erythema, eschar and edema observations gave the ‘total skin response score’ (minimum score=0, maximum score=10).

Table 4 shows the results of the three groups after 3 courses of PDT.

TABLE 4

| | GROUP 1 | GROUP 2 | GROUP 3 | GROUP 4 |
|---|---|---|---|---|
| Median Score | 0.1 mg/ml | 0.2 mg/ml | 1.0 mg/ml | Control |
| Black Tattoo Response | 4.5 | 3.5 | 3.5 | 1.5 |
| Green Tattoo Response | 3.5 | 3.5 | 5 | 1.5 |
| Skin Response* | 5.5 | 6 | 6 | 0 |

*maximal reaction over 3 treatments.

As can be seen the PDT caused fading in all cases with an acceptable skin response.

### Example 2

A tattooed human male having skin type II is given a skin photosensitivity test on skin area near the tattoo. No adverse skin reaction is observed. The skin over the tattooed area is shaved and the surface area estimated to be 3cm².

QLT0074 for Injection is reconstituted with Water for Injection to give a stock concentration of 2.0 mg/ml and diluted with 5% Dextrose in water to a concentration of 0.2 mg/ml. The skin surface is cleaned and alcohol-disinfected. 30 intradermal injections are given using a syringe and a 30 gauge ½ long needle. The injections are at a depth of approximately 3 mm and spaced evenly across the tattoo. The total volume of composition injected is 0.5 mL. The skin is then wiped with gauze to remove any excess drug.

A template mimicking the tattooed area is applied on skin to limit the light exposure to the target area. Fifteen minutes after injection of the drug, the skin is exposed to 10 J/cm² of LED-generated red light (688 nm-Q-100 LED Panel (Quantum Devices Inc, Barneveld Wis., USA)) at 75 mW/cm².

### Example 3

A tattooed human female having skin type II is given a skin photosensitivity test on skin area near the tattoo. No adverse skin reaction is observed. The skin over the tattooed area is shaved and the surface area estimated to be 4.5 cm².

A Macroflux® transdermal patch is treated with topical photosensitizer ointment (comprising 0.2 wt % lemuteporfin, 50 wt % PEG-200, 24 wt % Transcutol®, 10 wt % PEG-3350 and 15.8 wt % oleyl alcohol) and then applied to the tattooed area. The Macroflux patch incorporates a thin titanium screen with microprojections that, when applied to the skin, creates superficial pathways through the skin's barrier layer allowing penetration of the photosensitizer. The patch is left in place for 1-2 hrs and then removed. Any excess photosensitizer is wiped away.

A template mimicking the tattooed area is applied on skin to limit the light exposure to the target area. The skin is then exposed to 15 J/cm² of LED-generated red light (688 nm-Q-100 LED Panel (Quantum Devices Inc, Barneveld Wis., USA)) at 75 mW/cm².

### Example 4

A tattooed human male having skin type II is given a skin photosensitivity test on skin area near the tattoo. No adverse skin reaction is observed. The skin over the tattooed area is shaved and the surface area estimated to be 4cm².

The tattooed area is treated with the Althea PassPort system and then topical photosensitizer ointment comprising 0.2 wt % lemuteporfin, 50 wt % PEG-200, 24 wt % Transcutol®, 10 wt % PEG-3350 and 15.8 wt % oleyl alcohol is applied. The ointment is left for 1-2 hrs and then any excess is wiped away.

A template mimicking the tattooed area is applied on skin to limit the light exposure to the target area. The skin is then exposed to 10 J/cm² of LED-generated red light (688 nm-Q-100 LED Panel (Quantum Devices Inc, Barneveld Wis., USA)) at 75 mW/cm².

1. A photodynamic method of treating tattoos comprising the steps of:
(a) intradermally and/or locally delivering photosensitizer into tattooed target tissue; and
(b) irradiating the target tissue with activation energy at a wavelength appropriate to activate the photosensitizer.

2. The method of claim 1 wherein said delivering is intradermally.

3. The method of claim 1 wherein said delivering is locally.

4. The method of claim 1 wherein steps (a) and (b) are repeated two or more times.

5. The method of claim 4 wherein at least one week is left between the repeat treatments of steps (a) and (b).

6. The method of claim 1 wherein the photosensitizer is one or more of a porphyrin precursor or derivative thereof, a porphyrin or derivative thereof, a tetrahydrochlorin, a purpurin, a porphycene, a phenothiazinium, a bacteriochlorophyll, or combinations thereof.

7. The method of claim 6 wherein the porphyrin precursor is 5-amino-levulinic acid.

8. The method of claim 6 wherein the photosensitizer is a green porphyrin and/or a combination of a green porphyrin with another photosensitizer.

9. The method of claim 8 wherein the green porphyrin is verteporfin or QLT0074 and/or combinations thereof.

10. The method of claim 1 wherein the activation energy has a wavelength of from about 400 nm to about 800 nm.

11. The method of claim 1 wherein the total dose of activation energy is from about 0.1 J/cm² to about 100 J/cm².

12. The method of claim 1 wherein the irradiation step lasts from about 10 seconds to about 4 hours.

13. The method of claim 1 wherein the tattoo is faded by at least 50% after the course of treatment.
